SEQUENCE LISTING (1 sequence)

SEQ ID NO: 1 - protein, 13 aa, synthetic construct
CTVALPGGYVRVC

(19) United States
(12) Patent Application Publication
(10) Pub. No.: US 2024/0216555 A1
(43) Pub. Date: Jul. 4, 2024

(54) TARGETING AND TREATMENT OF CANCEROUS CELLS USING RADIOLABELED GRP78-BINDING AGENTS

(71) Applicant: University of Kentucky Research Foundation, Lexington, KY (US)

(72) Inventor: Vivek M. Rangnekar, Nicholasville, KY (US)

(21) Appl. No.: 18/545,717

(22) Filed: Dec. 19, 2023

Related U.S. Application Data
(60) Provisional application No. 63/477,880, filed on Dec. 30, 2022.

(51) Int. Cl. A61K 51/10

(52) U.S. Cl. CPC A61K 51/1072; A61K 2121/00

(57) ABSTRACT

Methods for treating cancer are provided and include administering a radiolabeled 78-kDa glucose-regulated-protein (GRP78) binding agent (GRP78-binding agent) to a subject in need thereof. Methods for inhibiting prostate cancer growth in a subject and compositions which make use of a radiolabeled GRP78-binding agent are also provided.

### CROSS-REFERENCE TO RELATED APPLICATION

The present application claims priority to U.S. Patent Application Ser. No. 63/477,880, filed on Dec. 30, 2022, the entire disclosure of which is incorporated herein by reference.

### REFERENCE TO AN ELECTRONIC SEQUENCE LISTING

The contents of the electronic sequence listing
(13177N_2690 US_SequenceListing.xml; Size: 1,960 bytes; and Date of Creation: Dec. 13, 2023) is herein incorporated by reference in its entirety.

### TECHNICAL FIELD

The presently disclosed subject matter generally relates to the targeting of cancerous cells using radiopharmaceuticals. In particular, the presently disclosed subject matter relates to methods for treating cancer and compositions that make use of a 78-kDa glucose-regulated protein (GRP78) binding agent (GRP78-binding agent).

### BACKGROUND

Prostate cancer (PCa) is the second leading cause of cancer related death in men in the United States.¹ Activation of the androgen receptor (AR) pathway by androgen constitutes a central axis contributing to PCa.²⁶ The AR signaling pathway, acting via a broad spectrum of downstream genes, sustains the growth and survival of prostate tumors. Androgen ablation therapy (also known as androgen-deprivation therapy or ADT) that directly targets the androgen signaling pathway is therefore the first line of treatment for prostate cancer. Prostate tumors can, however, become resistant to ADT and develop into castration-resistant prostate cancer (CRPC). Thus, while the five-year survival in PCa patients on ADT is over 90%, a significantly large number of patients progress to CRPC within a few years of therapy.²⁻⁴ Combination therapies have now become the standard of care treatment for metastatic PCa, but the efficacy of novel androgen receptor (AR) targeted therapies or chemotherapy as a subsequent line of treatment is short-lived.⁵

Several second-generation treatments, including the antiandrogen enzalutamide, CYP-17A1-inhibitor abiraterone, vaccine therapy with Sipuleucel-T, α-emitter 223Ra, or cytotoxic drugs such as docetaxel and cabazitaxel are currently available for CRPC.^{27,28} These treatments improve the lifespan of the patients, yet are not curative and the tumors develop resistance, with subsequent progression to metastatic CRPC (mCRPC).^{27,28} Such advanced prostate tumors claim more than 300,000 lives worldwide every year.²⁹.

Recently, the Federal Drug Administration (FDA) approved ¹⁷⁷Lu-PSMA-617 for men with mCRPC, but the clinical study on which this approval was granted indicated that approximately 40% of the patients did not respond to this treatment.⁶

Accordingly, there remains a need for new approaches to inhibit PCa progression. As such, novel treatments and pharmaceutical compositions which target PCa cells and inhibit PCa progression would thus be both beneficial and desirable.

### SUMMARY

The presently disclosed subject matter meets some or all of the above-identified needs, as will become evident to those of ordinary skill in the art after a study of information in this document.

This Summary describes several embodiments of the presently disclosed subject matter and, in many cases, lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features or all embodiments disclosed herein.

The presently disclosed subject matter includes methods for treating cancer and pharmaceutical compositions. In particular, the presently disclosed subject matter relates to methods for treating cancer and compositions that make use of a radiolabeled GRP78-binding agent.

In some embodiments, a method for treating cancer includes administering a radiolabeled GRP78-binding agent to a subject in need thereof. In some embodiments, the cancer treated is characterized, at least in part, by cancerous cells in which GRP78 is present in the plasma membrane of the cancerous cells. In some embodiments the GRP78-binding agent of the radiolabeled GRP78-binding agent includes a peptide comprising the sequence of SEQ ID NO: 1, or a functional fragment thereof. In some embodiments, the radiolabeled GRP78-binding agent is a radiolabeled peptide. In some embodiments, the radiolabeled peptide is a cyclic peptide. In some embodiments, the GRP78-binding agent is radiolabeled with Lutetium-177 (¹⁷⁷Lu). In some embodiments, the GRP78-binding agent is radiolabeled with Gallium-68 (⁶⁸Ga).

In some embodiments the cancer treated is prostate cancer. In some embodiments, the prostate cancer treated is castration-resistant prostate cancer (CRPC). In some embodiments, administering the radiolabeled GRP78-binding agent induces apoptotic cell death in prostate cancer cells in the subject. In some embodiments, administering the radiolabeled GRP78-binding agent induces apoptotic cell death in prostate cancer cells expressing tumor protein p53 (p53). In some embodiments, the cancer treated is cancer that is resistant to enzalutamide. In some embodiments, the cancer treated is metastatic cancer. In some embodiments, the cancer treated is metastatic castration-resistant prostate cancer (mCRPC). In some embodiments, administering the radiolabeled GRP78-binding agent induces endoplasmic reticulum (ER) stress and activates deoxyribonucleic acid (DNA) damage response (DDR) in cancerous tissue including cell-surface GRP78 (csGRP78) in the subject. In some embodiments, administering the radiolabeled GRP78-binding agent inhibits cancer growth in the subject.

Further provided, in some embodiments of the presently disclosed subject matter, are methods for inhibiting prostate cancer growth in a subject by administering a radiolabeled GRP78-binding agent to a subject in need thereof. In some of these additional embodiments, administration of the radiolabeled GRP78-binding agent induces ER stress and activates DDR in cancerous tissue in the subject. In some embodiments, the GRP78-binding agent of the radiolabeled GRP78-binding agent includes a peptide comprising the sequence of SEQ ID NO: 1, or a functional fragment thereof. In some embodiments, the radiolabeled GRP78-binding agent is a radiolabeled peptide. In some embodiments the radiolabeled peptide is a cyclic peptide. In some embodiments, the GRP78-binding agent is radiolabeled with Lutetium-177 (¹⁷⁷Lu). In some embodiments, the GRP78-binding agent is radiolabeled with Gallium-68 (⁶⁸Ga). In some embodiments, the prostate cancer is CRPC. In some embodiments, the prostate cancer is mCRPC. In some embodiments, administration of the GRP78-binding agent induces apoptotic cell death in prostate cancer cells expressing p53.

Still further provided, in some embodiments of the presently disclosed subject matter are pharmaceutical compositions which include a radiolabeled GRP78-binding agent and a pharmaceutically-acceptable carrier.

Further features and advantages of the presently disclosed subject matter will become evident to those of ordinary skill in the art after a study of the description, figures, and non-limiting examples in this document.

### BRIEF DESCRIPTION OF THE DRAWINGS

FIG. 1 is a diagram showing the activation of cross-talk between ER-stress and DDR in a cancerous tissue in response to the binding of radiolabeled peptides with csGRP78 of a cancerous tissue.

### BRIEF DESCRIPTION OF THE SEQUENCE LISTING

SEQ ID NO: 1 is an amino acid sequence of an embodiment of a peptide which binds to csGRP78.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom.

It will be understood that various details of the presently disclosed subject matter can be changed without departing from the scope of the subject matter disclosed herein. Furthermore, the description provided herein is for the purpose of illustration only, and not for the purpose of limitation.

Additionally, while the terms used herein are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the presently disclosed subject matter belongs. Although many methods, devices, and materials similar or equivalent to those described herein can be used in the practice or testing of the presently disclosed subject matter, representative methods, devices, and materials are now described.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Furthermore, following long-standing patent law convention, the terms “a”, “an”, and “the” refer to “one or more” when used in this application, including the claims. Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the abbreviations for any protective groups, amino acids and other compounds, are, unless indicated otherwise, in accord with their common usage, recognized abbreviations, or the IUPAC-IUB Commission on Biochemical Nomenclature.

Wherever any of the phrases “for example,” “such as,” “including” and the like are used herein, the phrase “and without limitation” is understood to follow unless explicitly stated otherwise. Similarly “an example,” “exemplary” and the like are understood to be non-limiting.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

GRP78 is an endoplasmic reticulum (ER) resident protein that is expressed at elevated levels in PCa cells.⁷⁻⁹ Although GRP78 is primarily resident in the ER in normal cells, it translocates to the plasma membrane in PCa, as well as in other cancer cells, and plays a critical role in disease progression, metastasis, and resistance to treatment.^{13, 14, 30, 31, 32} Such translocated GRP78 may be characterized as cell-surface GRP78 (csGRP78). Interestingly, the interplay of AR and oncoprotein Myc that contributes to CRPC progression is associated with GRP78 upregulation.^{8,10,11,12} Increased levels of GRP78 in the ER of cancer cells correlate with csGRP78 elevation.¹³⁻¹⁵ csGRP78 is also elevated in PCa cells (such as PC-3 cells) lacking a functional AR signaling pathway, implying that csGRP78 may serve as a target in neuroendocrine PCa.^{13,31} It is particularly interesting to note that csGRP78 operates as a receptor for diverse ligands that induce tumor growth, survival, or apoptosis.³³ Targeting the amino-terminal region of csGRP78 by the tumor suppressor Par-4, or the carboxy-terminal region of csGRP78 with a monoclonal antibody, induces cell death by apoptosis.^{13, 34, 35} Thus, csGRP78 is a targetable receptor on the surface of PCa cells.¹⁴ Moreover, the robust outcomes of csGRP78 targeting by CAR-T cells were recently demonstrated in acute myeloid leukemia (AML).³⁶

It has been shown that ligand binding to csGRP78 or exposure to ionizing radiation can trigger ER-stress leading to further increase in csGRP78 expression.^{13, 22, 37, 38} Under basal conditions, the master regulator GRP78 binds to the ER-resident proteins PERK, IRE1 and ATF6 and holds them in an inactive state.^{37,38} Accumulation of unfolded proteins in the ER, leads to ER-stress. This activates GRP78 to dissociate from PERK, IRE1 and ATF6 and bind to the unfolded proteins to resolve the ER-stress. Activation of PERK, IRE1, and ATF6 downstream signaling pathways enhances cell survival under ER-stress by facilitating ER protein folding and reducing misfolded protein levels by ER-associated protein degradation. However, when ER-stress is overwhelming and cannot be resolved, PERK and its downstream mediator ATF4 activate death signaling pathways.^{37,38} It has been shown that dominant-negative PERK inhibits apoptosis induced by binding of secreted pro-apoptotic protein Par-4 to csGRP78.¹³ PERK is a ubiquitously expressed serine/threonine kinase that autophosphorylates under ER-stress to activate its catalytic kinase activity and phosphorylate the eukaryotic translation initiator factor-2 (eIF2), thereby attenuating general protein synthesis. This allows the translation of specific mRNAs such as that coding for the ATF4 transcription factor, which promotes survival by NRF2 activation.^{39, 40} Under uncontrolled ER-stress, ATF4 engages the apoptotic program through expression of CHOP/GADD153 and PUMA pro-apoptotic proteins. Interestingly, radiation not only activates the ER-stress pathway but also activates the DDR pathway including ATM and ATR kinases that phosphorylate their target transducers Chk1, Chk2 and p53. Similar to ER-stress, the biological basis of DDR signaling is to provide the cells adequate time to repair the damage and recover, but to induce cell death in the case of irreparable damage.⁴¹⁻⁴³ The tumor suppressor p53 is believed to regulate the growth arrest or cell death outcome of DNA damage.⁴³ However, p53 is often mutated or deleted in advanced PCa, and the master regulators for p53-independent growth arrest or cell death are not fully elucidated.

The presently disclosed subject matter provides therapeutic options that includes the advantages of cancer directed radiation treatment and targeting of GRP78, a critical component of the signaling cross-talk of AR and oncogenic Myc.^{10,11} The methods and pharmaceutical compositions presented herein are based, at least in part, on the hypothesis that the cross-talk between the ER-stress pathway activated by cell-penetrating csGRP78-targeting peptide, and DNA damage activated by the radionuclide conjugated to the GRP78-targeting peptide will induce apoptotic cell death in PCa cells that express wild type p53 (LNCaP and C4-2 cells),^{44, 45} as well as in those that lack p53 (PC3 cells)⁴⁵ or express mutant p53 (CWR22Rv1 cells).⁴⁶ Accordingly, new methods and pharmaceutical compositions which utilize a radiolabeled GRP78-binding agent are disclosed herein.

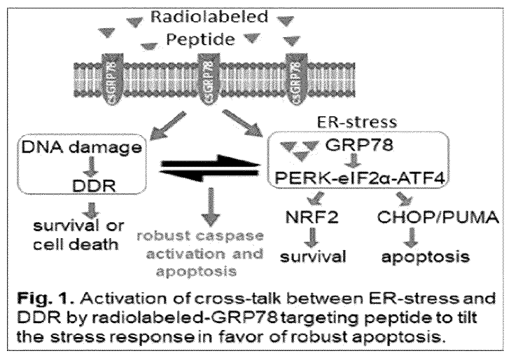

The presently disclosed subject matter includes methods for treating cancer in which a radiolabeled GRP78-binding agent is administered to a subject with cancer to promote cellular activity that results in cancer growth inhibition. In particular, embodiments of the presently disclosed subject matter include methods for treating cancer in which a radiolabeled GRP78-binding agent is administered to a subject to induce cross-talk between ER stress and DDR pathways to trigger extreme stress, amplification of cell death signal, and cancerous cell growth inhibition (FIG. 1). In some embodiments, the cancer treated is characterized, at least in part, by cancerous cells in which GRP78 is present in the plasma membrane of the cancerous cells. In some embodiments, the cancer treated is prostate cancer. In some embodiments, the cancer treated is a cancer resistant to treatment with enzalutamide (i.e., enzalutamide-resistant cancer), castration-resistant prostate cancer (CRPC), and/or metastatic cancer. Identification of the cancers specified herein can be achieved using diagnostic techniques readily known in the art. For instance, prostate cancer can be detected by digital rectal examination, Prostate Specific Antigen (PSA) testing using blood/serum samples obtained from a subject, tumor biopsy followed by histology analysis by a certified pathologist, and/or via computerized tomography (CT) scan or magnetic resonance imaging (MRI). In some embodiments, the methods disclosed herein may further involve diagnosing or otherwise identifying a subject as having one or more of the various cancers specified herein.

As will be appreciated by one of ordinary skill in the art upon study of the present document, the terms “treat,” “treatment,” and the like refer to the medical management of a subject with the intent to cure, ameliorate, stabilize, or prevent an infection or disease. This term includes treatment that is directed toward: direct treatment directed to the improvement of an infection state or disease state; causal treatment directed to the removal of the cause of an infection or disease; palliative treatment directed to the relief or amelioration of symptoms of the infection or disease, preventative or prophylactic treatment directed to minimizing or partially or completely inhibiting a disease or development of an associated disease; and supportive treatment that is directed to supplementing another specific therapy directed toward treatment of an infection or disease.

In some embodiments, administration of the radiolabeled GRP78-binding agent induces apoptotic cell death in prostate cancer cells, such as cells expressing tumor protein p53 (p53), in the subject. Where reference is made to a cell expressing p53, it is appreciated that such reference encompasses the cell expressing wild-type p53, mutant p53, or both wild-type p53 and mutant p53, unless specified otherwise. Reference to a cell expressing p53 is inclusive of the cell expressing wild-type p53 and/or mutant p53, unless specified otherwise. In some embodiments, administration of the radiolabeled GRP78-binding agent induces ER stress and activates DDR in a cancerous tissue including csGRP78 in the subject. In some embodiments, administration of the radiolabeled GRP78-binding agent inhibits cancer growth in the subject.

In some embodiments, the radiolabeled GRP78-binding agent comprises a peptide which includes a sequence of amino acids that facilitate binding of the radiolabeled peptide to csGRP78 of cancerous tissue within the subject, and which is radiolabeled with a radioactive isotope. The terms “radioactive isotope” and “radionuclide” are used interchangeably herein. In some embodiments, the peptide of the radiolabeled GRP78-binding agent is a cyclic peptide. Without wishing to be bound to any particular theory, the use of a cyclic peptide is believed to be beneficial as the cyclic structure of the peptide is anticipated to provide more precise binding and avidity for csGRP78.

In some embodiments, the radiolabeled GRP78-binding agent includes a peptide comprising the sequence of SEQ ID NO: 1 (i.e., CTVALPGGYVRVC), or fragments, and/or variants thereof. The term “fragment” when used in relation to a reference peptide, refers to a peptide in which amino acid residues are deleted as compared to the reference peptide itself, but where the remaining amino acid sequence is usually identical to the corresponding positions in the reference peptide. Such deletions may occur at the amino-terminus of the reference peptide, the carboxy-terminus of the reference peptide, or both. Peptide fragments can also be inclusive of “functional fragments,” in which case the fragment retains some or all of the activity of the reference peptide. Accordingly, where reference is made to a “functional fragment” of a peptide of the radiolabeled GRP78-binding agent, it is appreciated that such functional fragment is capable of binding with csGRP78.

The term “variant,” as used herein in relation to a reference peptide, refers to an amino acid sequence that is different from the reference peptide by one or more amino acids. In some embodiments, a variant peptide may differ from a reference peptide by one or more amino acid substitutions. For example, a peptide variant can differ from the peptide of SEQ ID NO: 1 by one or more amino acid substitutions, i.e., mutations. In this regard, peptide variants comprising combinations of two or more mutations can respectively be referred to as double mutants, triple mutants, and so forth. It will be recognized that certain mutations can result in a notable change in function of a peptide, while other mutations will result in little to no notable change in function of the peptide.

In some embodiments, the radiolabeled GRP78-binding agent may comprise a peptide that shares 100% homology with the peptide of SEQ ID NO: 1. In some embodiments, the radiolabeled GRP78-binding agent may comprise a peptide that shares at least 95% homology with the peptide of SEQ ID NO: 1. In some embodiments, the radiolabeled GRP78-binding agent may comprise a peptide that shares at least 90% homology with the peptide of SEQ ID NO: 1. In some embodiments, the radiolabeled GRP78-binding agent may comprise a peptide that shares at least 85% homology with the peptide of SEQ ID NO: 1. In some embodiments, the radiolabeled GRP78-binding agent may comprise a peptide that shares at least 80% homology with the peptide of SEQ ID NO: 1. In some embodiments, the radiolabeled GRP78-binding agent may comprise a peptide that shares at least 75% homology with the peptide of SEQ ID NO: 1.

Although the radiolabeled GRP78-binding agent is sometimes referred to herein as a radiolabeled peptide, it should be appreciated that embodiments are also contemplated in which the radiolabeled GRP78-binding agent is a radiolabeled antibody, which includes a chain region including an amino acid sequence (e.g., SEQ ID NO: 1) that facilitates binding of the radiolabeled antibody to csGRP78 of cancerous tissue within the subject. In this regard, embodiments in which the radiolabeled GRP78-binding agent is a radiolabeled antibody including a chain region with one or more peptides consistent with those presently disclosed are also contemplated herein.

The radiolabeled GRP78-binding agent includes one or more radioactive isotopes which can be traced for pharmacokinetics/biodistribution studies and/or serve as a therapeutic payload which promotes cellular activity resulting in cancer growth inhibition (e.g., inhibition of tumor growth or metastasis). The various GRP78-binding agent peptides disclosed herein can be radiolabeled with radioactive isotopes utilizing techniques known in the art, such as those used for radiolabeling prostate-specific membrane antigen (PSMA) targeting agents, as disclosed, e.g., in Carpanese, et al.⁵⁰ In some embodiments, the GRP78-binding agent is radiolabeled with Lutetium-177 (¹⁷⁷Lu). In some embodiments, the GRP78-binding agent is radiolabeled with Gallium-68 (⁶⁸Ga). Although the GRP78-binding agent is primarily referenced herein as being radiolabeled with ¹⁷⁷Lu or ⁶⁸Ga, embodiments in which the GRP78-binding agent is radiolabeled with other isotopes with a short half-life are also contemplated herein. For instance, in some embodiments and implementations the GRP78-binding agent may be radiolabeled with Copper-64 (⁶⁴Cu), a positron and beta emitting isotope of copper, and utilized for bioavailability studies.

The presently disclosed subject matter also includes methods for inhibiting prostate cancer (PCa) growth that involves administering a radiolabeled GRP78-binding agent to a subject with prostate cancer. In some embodiments, the prostate cancer treated is CRPC, enzalutamide resistant cancer, and/or metastatic cancer. In some embodiments, following administration of the radiolabeled GRP78-binding agent to the subject, inhibition of PCa growth within the subject occurs as a result of the radiolabeled GRP78-binding agent binding to a cell-surface GRP78 of a cancerous tissue in the subject and, consequently, inducing ER stress and activating DDR damage in the cancerous tissue.

The terms “inhibit” and “inhibiting” do not necessarily refer to the ability to completely eliminate an infection, disease, or characteristic or symptom thereof, but, rather, are inclusive of elimination, reduction, and restriction of further progression of an infection, disease, or characteristic or symptom thereof. Thus, such terms do not imply complete elimination or a particular degree of reduction of an infection, disease, or characteristic or symptom thereof. Instead, the terms can be used to refer to a result that is less than 100% reduction relative to a control. Thus, in some embodiments, the terms can be used to refer to a reduction in an infection, disease, or characteristic or symptom thereof by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% relative to a control that has not received the treatment.

The radiolabeled GRP78-binding agents administered to the subject in the methods of inhibiting prostate cancer growth are consistent with those described above with reference to the methods for treating cancer. That is, various embodiments of a method for inhibiting PCa growth are contemplated wherein the radiolabeled GRP78-binding agent in each respective embodiment corresponds to a different one of the radiolabeled GRP78-binding agent embodiments described above for use in the methods for treating cancer. For instance, in one exemplary embodiment, the radiolabeled GRP78-binding agent may include a peptide comprising the sequence of SEQ ID NO: 1, or a functional fragment thereof, that is radiolabel with ¹⁷⁷Lu or ⁶⁸Ga. In some embodiments, administration of the radiolabeled GRP78-binding agent induces apoptotic cell death in prostate cancer cells, such as prostate cancer cells expressing tumor protein p53, in the subject.

The presently disclosed subject matter further includes and makes use of pharmaceutical compositions comprising the radiolabeled GRP78-binding agents described herein as well as a pharmaceutically-acceptable carrier. Various pharmaceutical composition embodiments are contemplated, wherein each respective embodiment includes a different one of the radiolabeled GRP78-binding agents described herein and a pharmaceutically-acceptable carrier. For instance in some embodiments, the pharmaceutical composition may comprise a peptide comprising the sequence of SEQ ID NO: 1, or a functional fragment thereof, that is radiolabeled with ¹⁷⁷Lu or ⁶⁸Ga.

The term “pharmaceutically-acceptable carrier” as used herein refers to sterile aqueous or nonaqueous solutions, dispersions, suspensions, or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants, such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like.

The term “pharmaceutically-acceptable carrier” as used herein refers to sterile aqueous or nonaqueous solutions, dispersions, suspensions, or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants, such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like.

Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly (orthoesters) and poly(anhydrides). Depending upon the ratio of radiolabeled GRP78-binding agent to biodegradable polymer and the nature of the particular biodegradable polymer employed, the rate of polypeptide release can be controlled. Depot injectable formulations can also be prepared by entrapping the radiolabeled GRP78-binding agent in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations can further include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents.

The compositions can also take forms such as suspensions, solutions, or emulsions in oily or aqueous vehicles, and can contain formulatory agents such as suspending, stabilizing and/or dispersing agents. Alternatively, the polypeptides can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose, or gelatin-free binding agents); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration, the compositions can take the form of tablets or lozenges formulated in a conventional manner.

The compositions can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). The compounds can also be formulated in rectal compositions, creams or lotions, or transdermal patches.

In accordance with the presently-disclosed subject matter, the term “administering” refers to any method of providing the radiolabeled GRP78-binding agent and/or pharmaceutical composition including the same to a subject. Such methods are well known to those skilled in the art and include, but are not limited to, oral administration, transdermal administration, nasal administration, intracerebral administration, and administration by injection, which itself can include intravenous administration, intra-arterial administration, intramuscular administration, subcutaneous administration, intravitreous administration, intracameral (into anterior chamber) administration, and the like. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

In the methods disclosed herein, the radiolabeled GRP78-binding agent will typically be administered to a subject in an effective amount. As used herein, the terms “effective amount” refers to a dosage sufficient to provide treatment. The exact amount that is required will vary from subject to subject, depending on the species, age, and general condition of the subject, the particular carrier or adjuvant being used, mode of administration, and the like. As such, the effective amount will vary based on the particular circumstances, and an appropriate effective amount can be determined in a particular case by one of ordinary skill in the art using only routine experimentation. The radiolabeled GRP78-binding agent may be administered in conjunction with a pharmaceutically-acceptable carrier, e.g, as part of a pharmaceutical composition.

The present methods can be performed on a wide variety of subjects. Indeed, the term “subject” as used herein is also not particularly limited. The term “subject” is inclusive of vertebrates, such as mammals, and the term “subject” can include human and veterinary subjects. Thus, the subject of the herein disclosed methods can be a human, non-human primate, horse, pig, rabbit, dog, sheep, goat, cow, cat, guinea pig, rodent, or the like. The term does not denote a particular age or sex. Thus, adult and newborn subjects, as well as fetuses, whether male or female, are intended to be covered.

The presently-disclosed subject matter is further illustrated by the following specific but non-limiting prophetic examples.

### EXAMPLES

The presently disclosed subject matter is further illustrated by the forthcoming studies described below, in which: the efficacy of a radiolabeled GRP78-targeting peptide to induce apoptosis in CRPC by co-activating ER Stress and DNA damage pathways will be examined; quantitative biodistribution analysis of the radiolabeled peptide will be undertaken through pharmacokinetic and positron emission tomography (PET) imaging studies in mice with xenografts; and tumor growth and metastasis inhibition will be tested using preclinical models.

The forthcoming studies propose a novel strategy that combines the activation of DNA damage response (DDR) by radiation coupled with an endoplasmic reticulum (ER) stress response to cause growth inhibition of PCa. Acute activation of the ER stress response pathway or DDR signaling pathway typically induces growth arrest and allows the cells to repair their damage and recover. It is anticipated that coupled activation of the ER stress and DDR pathways will induce extreme stress and irreparable damage to cause cell death and inhibition of tumor growth. This approach involves targeting cell-surface GRP78 (csGRP78) that is elevated in PCa with a cyclic-peptide that is cell-penetrating and reaches the ER to induce ER-stress. As this csGRP78-binding peptide is not expected to cause cell death on its own, the beta emitter ¹⁷⁷Lu radiolabel will be attached to it as a payload to cause DNA damage in tumors. By co-inducing ER-stress and DDR, the ¹⁷⁷Lu-labeled csGRP78-binding peptide will trigger extreme stress and cell death. The strategy of targeting csGRP78 is particularly applicable to the majority of prostate tumors as interactions between AR and oncogenic MYC protein that contribute to progression of PCa to CRPC result in upregulation of GRP78 levels in the ER, and elevation of csGRP78 correlates with increased ER levels of GRP78. For pharmacokinetics/biodistribution studies in tumor bearing mice, the peptide will be labeled with ⁶⁸Ga and traced by micro-PET imaging. The therapeutic outcome of inducing ER-stress, DNA damage, and cell death proteins will be validated in cell culture and tumor xenografts by using ¹⁷⁷Lu-radiolabeled-peptide. Together with immunohistochemistry for ER-stress, DDR, and cell survival or cell death associated proteins, and digital image analysis, these studies will allow comparison of the effect of targeting csGRP78 with the radiolabeled-peptide on cell fate (i.e., survival or cell death) in normal and tumor tissues. Development of resistant clones, if any, will be monitored to determine whether proteins critical for ER-stress, DDR and cell death are differentially expressed in resistant tumors to tilt the outcome toward cell survival relative to those tumors that undergo cell death and growth inhibition. The findings will provide the biological and mechanistic framework for future studies on csGRP78-targeting radiopharmaceuticals for robust growth inhibition of prostate tumors.

In the studies, a cyclic peptide of the sequence CTVALPGGYVRVC (SEQ ID NO: 1) that has been previously shown to bind to csGRP78 and translocate to the ER^{16,17} will be used. Radiolabeled-derivatives of the csGRP78-targeting peptide using the positron (1+) emitting ⁶⁸Ga tracer (18-20) to enable biodistribution and imaging studies, or electron (1) particle emitting ¹⁷⁷Lu (21) will be generated to evaluate gene expression and tumor growth inhibition. The radiolabeled-peptide is expected to bind csGRP78, translocate into the ER, and induce ER-stress that can promote feed-forward upregulation of csGRP78.^{13,16,17} ER-stress activation by itself may not be sufficient to induce cell death. On the other hand, ionizing radiation induces ER-stress, elevates csGRP78 in cancer cells, and activates DNA damage response (DDR).²²⁻²⁴ The ¹⁷⁷Lu-labeled peptide is therefore expected to induce cross-talk between the ER-stress and DDR pathways, thereby triggering extreme stress, amplification of the cell death signal, and tumor growth inhibition (FIG. 1). The proposed studies will test the hypothesis that the ¹⁷⁷Lu-labeled, cell-penetrating, peptide that targets csGRP78 will induce co-parallel ER-stress and DDR, resulting in robust caspase activation, apoptosis and growth inhibition of CRPC.

### Determining the Biodistribution of the GRP78-Targeting Radiolabeled-Peptide and Test for Activation of Key Proteins in the ER-Stress, DDR, and Apoptosis Pathways in Xenografts

Biodistribution and imaging studies with the ⁶⁸Ga-labeled peptide will be performed in mice carrying xenografts derived from enzalutamide-resistant PCa cells. Although the positron (1+) emitting isotope ⁶⁸Ga is considered safe for non-invasive and quantitative imaging, its cytotoxicity has been recently reported in PCa cell culture.²⁵ Immunohistochemistry (IHC) and digital image analysis will therefore be performed to determine whether the ⁶⁸Ga labeled-peptide induces ER-stress, DDR, and apoptosis pathways in normal and tumor tissues.

### Determine the Growth Inhibitory Effect of the Radiolabeled GRP78-Targeting Peptide on Tumor Xenografts in Mouse Models

Whether treatment with the ¹⁷⁷Lu-labeled GRP78-targeting peptide can inhibit the growth of enzalutamide-resistant prostate tumor xenografts will be tested. To determine the functional role of ER-stress in tumor growth inhibition, the outcome of knockdown of GRP78 and PERK, the proximal protein kinase activated following ER-stress, will be determined. Emergence of resistant clones, if any, will be monitored and ER-stress, DDR, and caspase activation in resistant tumors will be compared to that of tumors that favorably respond to the treatment.

The proposed studies will also test the hypothesis that a radiolabeled peptide or antibody that targets csGRP78 will overcome or bypass resistance to the currently available PCa treatments and inhibit the growth of mCRPC. Preclinical studies will utilize cell culture and mouse models to determine growth inhibition of CRPC and mCRPC by targeting csGRP78, with a radiolabeled-GRP78 binding peptide or radiolabeled-GRP78 carboxyl-terminus antibody. This approach will take advantage of the potential of radiation, emitted by the radiolabeled csGRP78 targeting moiety, to further upregulate csGRP78 on the cancer cells. Moreover, as the expression of csGRP78 may be potentially low in some tumors, the ALK/MET/ROS1 inhibitor Crizotinib, as a repurposed drug, will be used to elevate csGRP78 levels in PCa cells but not in normal cells. The studies will include the PCa-targeting agent Lu-177-PSMA-617, which has been recently FDA-approved for PCa treatment, as an internal reference to compare the relative efficacy of the csGRP78-targeting strategy. All of these radiopharmaceuticals will be generated at the Beckman Research Institute of the City of Hope, Duarte, CA, and tested in the presence or absence of Crizotinib by a collaborative team of investigators at the University of Kentucky, Lexington, KY, and Beckman Research Institute, City of Hope, Duarte, CA. The project will adhere to a protocol approved by the institutional committees for radioisotope labeling, safe overnight delivery and transportation from the Beckman Research Institute to the University of Kentucky, as well as personnel protection, animal use and waste disposal.

These studies will provide new insights into the downstream events activated by the co-parallel induction of ER-stress and DDR pathways, and help determine whether the ¹⁷⁷Lu-labeled GRP78-targeting peptide can trigger extreme stress leading to apoptosis and tumor growth inhibition in enzalutamide-resistant CRPC cells.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Siegel R L, Miller K D, Fuchs H E, and Jemal A (2022) Cancer statistics, 2022. CA Cancer J Clin 72(1): 7-33.
2. Estébanez-Perpiña E, Bevan C L, and McEwan I J (2021) Eighty years of targeting androgen receptor activity in prostate cancer: The fight goes on. Cancers (Basel). 13(3): 509.
3. https://www.cancer.org/cancer/prostate-cancer/treating/hormone-therapy.html
4. Patrikidou A, Zilli T, Baciarello G Terisse S, Hamilou Z, and Fizazi K (2021) Should androgen deprivation therapy and other systemic treatments be used in men with prostate cancer and a rising PSA post-local treatments? Therapeutic Advances in Medical Oncology 13: 17588359211051870.
5. Davis I D (2022) Combination therapy in metastatic hormone-sensitive prostate cancer: Is three a crowd? Ther Adv Med Oncol. 14: 17588359221086827.
6. Sartor O, de Bono J, Chi K N, Fizazi K, Herrmann K, Rahbar K, et al. (2021) Lutetium-177-PSMA-617 for metastatic castration-resistant prostate cancer. N Engl J Med 385:1091-1103.
7. Luo B, and Lee A S (2012) The critical roles of endoplasmic reticulum chaperones and unfolded protein response in tumorigenesis and anticancer therapies. Oncogene 32(7): 805-818.
8. Tan S S, Ahmad I, Bennett H L, Singh L, Nixon C, Seywright M, et al. (2011) GRP78 up-regulation is associated with androgen receptor status, Hsp70-Hsp90 client proteins and castrate-resistant prostate cancer. J. Pathol. 223(1): 81-87.
9. Bailly C. and Waring M J (2019) Pharmacological effectors of GRP78 chaperone in cancers. Biochem Pharmacol. 163: 269-278.
10. Zhang T, Li N, Sun C, Jin Y, and Sheng X (2020) MYC and the unfolded protein response in cancer: Synthetic lethal partners in crime? EMBO Mol Med 12: e11845.
11. Qiu X, Boufaied N, Hallal T, Feit A, de Polo A, Luoma A M, et al. (2022) MYC drives aggressive prostate cancer by disrupting transcriptional pause release at androgen receptor targets. Nature Communications 13: article number 2559.
12. Pootrakul L, Datar R H, Shi S R, Cai J, Hawes D, Groshen S G, Lee A S, and Cote R J (2006) Expression of stress response protein Grp78 is associated with the development of castration-resistant prostate cancer. Clin Cancer Res 12(20): 5987-5993.
13. Burikhanov R, Zhao Y, Goswami A, Qiu S, Schwarze S R, and Rangnekar V M (2009) The tumor suppressor Par-4 activates an extrinsic pathway for apoptosis. Cell 138: 377-388.
14. Araujo N, Hebbar N, and Rangnekar V M (2018) GRP78 is a targetable receptor on cancer and stromal cells. EBioMedicine 33: 2-3.
15. Ibrahim I M, Abdelmalek D H, and Elfiky A A (2019) GRP78: A cell's response to stress. Life Sci 226:156-163.
16. Kim Y, Lillo A M, Steiniger S C J, Liu Y, Ballatore C, Anichini A, et al. (2006) Targeting heat shock proteins on cancer cells: selection, characterization, and cell-penetrating properties of a peptidic GRP78 ligand. Biochemistry 45(31): 9434-9444.
17. Yoneda Y, Steiniger S C J, Čapková K, Mee J M, Liu Y, Kaufmann G F, and Jandaa K D (2008) A cell-penetrating peptidic GRP78 ligand for tumor cell-specific prodrug therapy. Bioorg Med Chem Lett. 18(5): 1632-1636.
18. Hennrich U and Benešová M (2020) [68Ga]-DOTA-TOC: The first FDA-approved 68Ga-radiopharmaceutical for PET imaging. Pharmaceuticals (Basel) 13(3): 38.
19. Hu X, Wu Y, Yang P, Wang J, Wang P, and Cai J (2021) Performance of 68Ga-labeled prostate-specific membrane antigen ligand positron emission tomography/computed tomography in the diagnosis of primary prostate cancer: a systematic review and meta-analysis. Int Braz J Urol. 47: doi: 10.1590/S1677-5538.
20. Xu Y, Jiang J, Wang H, Yu W, and Sun G (2021) Synthesis and preclinical evaluation of [68 Ga]SP94 for micro-PET imaging of GRP78 expression in hepatocellular carcinoma. ACS Med Chem Lett 12(10):1553-1558.
21. Dash A, Raghavan M, Pillai A, and Knapp, F F J (2015) Production of 177Lu for targeted radionuclide therapy: available options. Nucl Med Mol Imaging. 49(2): 85-107.
22. Passarella R J, Spratt D E, van der Ende A E, Phillips J G, Wu H, Sathiyakumar V, Zhou L, Hallahan D E, Harth E, and Diaz R (2010) Targeted nanoparticles that deliver a sustained, specific release of Paclitaxel to irradiated tumors. Cancer Res 70(11):4550-4559.
23. Dadey D Y A, Kapoor V, Hoye K, Khudanyan A, Collins A, Thotala D, and Hallahan D E (2017) Antibody targeting GRP78 enhances the efficacy of radiation therapy in human glioblastoma and non-small cell lung cancer cell lines and tumor models. Clin Cancer Res. 23: 2556-2564.
24. Nickoloff J A, Boss M K, Allen C P, and LaRue S M (2017) Translational research in radiation-induced DNA damage signaling and repair. Transl Cancer Res. 6(Suppl 5): S875-S891.
25. Hioki T, Gholami Y H, Mckelvey K J, Aslani A, Marquis H, Eslick E M, Willowson K P, Howell V M and Bailey D L (2021) Overlooked potential of positrons in cancer therapy. Sci Rep. 11: 2475.
26. Bluemn E, and Nelson P S (2012) The androgen/androgen receptor axis in prostate cancer. Curr Opin Oncol. 24(3): 251-257.
27. Verma S, Prajapati K S, Kushwaha P P, Shuaib M, Singh A T, Kumar S, Gupta S (2020) Resistance to second generation antiandrogens in prostate cancer: pathways and mechanisms. Cancer Drug Resist 3:742-761.
28. Terrisse S, Karamouza E, Parker C C, Sartor A O, James N D, Pirrie S, et al. (2020) Overall survival in men with bone metastases from castration-resistant prostate cancer treated with bone-targeting radioisotopes—A meta-analysis of individual patient data from randomized clinical trials. JAMA Oncol. 6(2): 206-216.
29. Sung H, Ferlay J, Siegel R L, Laversanne M, Soerjomataram I, Jemal A, et al. (2021) Global cancer statistics 2020: GLOBOCAN estimates of incidence and mortality worldwide for 36 cancers in 185 countries. CA Cancer J Clin. 71:209-249.
30. Farshbaf M, Khosroushahi A Y, Mojarad-Jabali S, Zarebkohan A, Valizadeh H, Walker P R (2020) cell-surface GRP78: An emerging imaging marker and therapeutic target for cancer. J Control Release. 328:932-941.
31. Thakur G, Sathe G, Kundu I, Biswas B, Gautam P, Alkahtani S, Idicula-Thomas S, Sirdeshmukh R, Kishore U, and Madan T (2021) Membrane interactome of a recombinant fragment of human surfactant protein D reveals GRP78 as a novel binding partner in PC3, a metastatic prostate cancer cell line. Front. Immunol. 11: article number 600660.
32. Wang M, Wey S, Zhang Y, Ye R, and Lee A S (2009) Role of the unfolded protein response regulator GRP78/BiP in development, cancer, and neurological disorders. Antioxid Redox Signal. 11(9): 2307-2316.
33. Gonzalez-Gronow M, Gopal U, Austin R C, Pizzo S V (2021) Glucose-regulated protein (GRP78) is an important cell-surface receptor for viral invasion, cancers, and neurological disorders. IUBMB Life 73(6): 843-854.
34. Gopal U, Mowery Y, Young K and Pizzo S V (2019) Targeting cell-surface GRP78 enhances pancreatic cancer radiosensitivity through YAP/TAZ protein signaling. Journal of Biological Chemistry 294(38): 13939-13952.
35. Hernandez I, and Cohen M (2022) Linking cell-surface GRP78 to cancer: From basic research to clinical value of GRP78 antibodies. Cancer Letters 524: 1-14.
36. Hebbar N, Epperly R, Vaidya A, Thanekar U, Moore S E, Umeda M, Ma J, et al. (2022) CAR T cells redirected to cell-surface GRP78 display robust anti-acute myeloid leukemia activity and do not target hematopoietic progenitor cells. Nature Communications 13: article number: 587.
37. Rozpędek W, Pytel D, Mucha B, Leszczyńska H, Diehl J A, and Majsterek I (2016) The role of the PERK/eIF2a/ATF4/CHOP signaling pathway in tumor progression during endoplasmic reticulum stress. Curr Mol Med. 16(6): 533-544.
38. Metcalf M G, Higuchi-Sanabria R, Garcia G, Tsui C K, and Dillin A (2020) Beyond the cell factory: Homeostatic regulation of and by the UPR-ER. Science Advances 6: No. 29.
39. Cullinan S B and Diehl J A (2004) PERK-dependent activation of Nrf2 contributes to redox homeostasis and cell survival following endoplasmic reticulum stress. Journal of Biological Chemistry 279(19): 20108-20117.
40. He F, Ru X, and Wen T (2020) NRF2, a transcription factor for stress response and beyond. Int J Mol Sci. 21(13): 4777.
41. Harvey R F and Willis A E (2018) Post-transcriptional control of stress responses in cancer. Current Opinion in Genetics & Development 48:30-35.
42. González-Quiroz M, Blondel A, Sagredo A, Hetz C, Chevet E, and Pedeux R (2020) When endoplasmic reticulum proteostasis meets the DNA damage response. Trends Cell Biol 30(11):881-891.
43. Wang J Y J (2019) Cell death response to DNA damage. Yale J Biol Med. 92(4): 771-779.
44. Karanika S, Karantanos T, Li L, Wang J, Park S, Yang G, Zuo X, et al. (2017) Targeting DNA damage response in prostate cancer by inhibiting androgen receptor-CDC6-ATR-Chk1 signaling. Cell Reports 18: 1970-1981.
45. Chappell W H, Lehmann B D, Terrian D M, Abrams S L, Steelman L S, and McCubrey J A (2012) p53 expression controls prostate cancer sensitivity to chemotherapy and the MDM2 inhibitor Nutlin-3. Cell Cycle 11(24): 4579-4588.
46. Chopra H, Khan Z, Contreras J, Wang H, Sedrak A and Zhu Y (2018) Activation of p53 and destabilization of androgen receptor by combinatorial inhibition of MDM2 and MDMX in prostate cancer cells. Oncotarget 9: 6270-6281.
47. Liu C, Armstrong C, Zhu Y, Lou W, and Gao A C (2016) Niclosamide enhances abiraterone treatment via inhibition of androgen receptor variants in castration-resistant prostate cancer. Oncotarget 7(22):32210-20.
48. Chendil D, Das A, Dey S, Mohiuddin M, and Ahmed M M (2002) Par-4, A pro-apoptotic gene, inhibits radiation-induced NFκB activity and Bcl-2 expression leading to induction of radiosensitivity in human prostate cancer cells PC-3, Cancer Biology & Therapy 1(2): 152-160.
49. Burikhanov R, Hebbar N, Noothi S K, Shukla N, Sledziona J, Araujo N, Kudrimoti M, Wang Q J, Watt D S, Welch D R, Maranchie J, Harada A, Rangnekar V M (2017) Chloroquine-inducible Par-4 secretion is essential for tumor cell apoptosis and inhibition of metastasis. Cell Rep. 18(2):508-519.
50. Debora Carpanese, Guillermina Ferro-Flores, Blanca Ocampo-Garcia, Clara Santos-Cuevas, Nicola Salvarese, Mariangela Figini, Giulio Fracasso, Laura De Nardo, Cristina Bolzati, Antonio Rosato & Laura Meléndez-Alafort. Development of ¹⁷⁷Lu-scFvD2B as a Potential Immunotheranostic Agent for Tumors Overexpressing the Prostate Specific Membrane Antigen. Scientific Reports (2020) 10:9313. https://doi.org/10.1038/s41598-020-66285-2).

What is claimed is:

1. A method for treating cancer, comprising administering a radiolabeled 78-kDa glucose-regulated-protein (GRP78) binding agent (GRP78-binding agent) to a subject in need thereof, wherein the cancer is characterized, at least in part, by cancerous cells in which GRP78 is present in the plasma membrane of the cancerous cells.

2. The method of claim 1, wherein the GRP78-binding agent of the radiolabeled GRP78-binding agent includes a peptide comprising the sequence of SEQ ID NO: 1, or a functional fragment thereof.

3. The method of claim 3, wherein the radiolabeled GRP78-binding agent is a radiolabeled peptide.

4. The method of claim 1, wherein the radiolabeled GRP78-binding agent is a cyclic peptide.

5. The method of claim 1, wherein the GRP78-binding agent is radiolabeled with Lutetium-177 (¹⁷⁷Lu).

6. The method of claim 1, wherein the GRP78-binding agent is radiolabeled with Gallium-68 (⁶⁸Ga).

7. The method of claim 1, wherein the cancer is prostate cancer.

8. the method of claim 7, wherein the cancer is castration-resistant prostate cancer (CRPC).

9. The method of claim 7, wherein administering the radiolabeled GRP78-binding agent induces apoptotic cell death in prostate cancer cells.

10. The method of claim 9, wherein the prostate cancer cells express tumor protein p53.

11. The method of claim 7, wherein the cancer is resistant to enzalutamide.

12. The method of claim 7, wherein the cancer is metastatic cancer.

13. The method of claim 1, wherein administering the radiolabeled GRP78-binding agent induces endoplasmic reticulum (ER) stress and activates deoxyribonucleic acid (DNA) damage response (DDR) in a cancerous tissue including cell-surface GRP78 (csGRP78) in the subject.

14. The method of claim 1, wherein administering the radiolabeled GRP78-binding agent inhibits cancer growth in the subject.

15. A method for inhibiting prostate cancer growth in a subject, comprising administering a radiolabeled GRP78-binding agent to a subject in need thereof, wherein radiolabeled GRP78-binding agent, subsequent to binding to cell-surface GRP78 (csGRP78) of cancerous tissue in the subject, induces endoplasmic reticulum (ER) stress and activates deoxyribonucleic acid (DNA) damage response (DDR) in the cancerous tissue.

16. The method of claim 15, wherein the GRP78-binding agent of the radiolabeled GRP78-binding agent includes a peptide comprising the sequence of SEQ ID NO: 1, or a functional fragment thereof.

17. The method of claim 16, wherein the radiolabeled GRP78-binding agent comprises a peptide radiolabeled with a radionuclide selected from the group consisting of Lutetium-177 (¹⁷⁷Lu) and Gallium-68 (⁶⁸Ga).

18. The method of claim 17, wherein the peptide is a cyclic peptide that is radiolabeled with ¹⁷⁷Lu.

19. A pharmaceutical composition, comprising a radiolabeled GRP78-binding agent and a pharmaceutically-acceptable carrier.

20. The composition of claim 19, wherein the radiolabeled GRP78-binding agent comprises
a peptide comprising the sequence of SEQ ID NO: 1, or a functional fragment thereof, radiolabeled with a radionuclide selected from the group consisting of Lutetium-177 (¹⁷⁷Lu) and Gallium-68 (⁶⁸Ga).

21. The composition of claim 20, wherein the radionuclide is ¹⁷⁷Lu.
